(19) United States
(12) Patent Application Publication
(10) Pub. No.: US 2023/0107915 A1
(43) Pub. Date: Apr. 6, 2023

(54) METHOD AND APPARATUS FOR EDEMA DETECTION

(71) Applicants: ResMed Sensor Technologies Limited, Dublin (IE); ResMed Pty Ltd, Bella Vista NSW (AU)

(72) Inventors: Graeme Alexander Lyon, Dublin (IE); Luke Burraston, Bella Vista NSW (AU)

(21) Appl. No.: 17/802,490

(22) PCT Filed: Feb. 26, 2021
(86) PCT No.: PCT/IB2021/051590

§ 371 Date: Aug. 25, 2022

Related U.S. Application Data
(60) Provisional application No. 62/981,976, filed on Feb. 26, 2020.

(51) Int. Cl. A61B 5/00

(52) U.S. Cl. CPC A61B 5/4878; A61B 5/442; A61B 5/0077; A61B 5/7435

(57) ABSTRACT

A system has an electronic interface, one or more sensors, a memory, and a control system including one or more processors. The processors are configured to execute instructions for activating an edema test, automatically or in response to receiving a user request via the electronic interface. Upon activation, user instructions are provided to locate, via one or more of the sensors, a skin area in the subject for testing. A user is instructed to depress the skin area for causing a temporary indentation. One or more images of the temporary indentation are captured, via one or more of the sensors, over a period of time following the depression of the skin area by the user. The images are analyzed for characteristics of skin bounce-back, which represents rebounding of the skin area after the temporary indentation. An edema result is determined based on the characteristics of the skin bounce-back.

### CROSS-REFERENCE TO RELATED APPLICATIONS

This application claims the benefit of, and priority to, U.S. Provisional Patent Application Ser. No. 62/981,976, filed on Feb. 26, 2020, titled “Method and Apparatus for Edema Detection,” which is hereby incorporated by reference herein in its entirety.

### TECHNICAL FIELD

The present disclosure relates generally to systems and methods for edema testing, and more particularly, to systems and methods for analyzing characteristics of skin bounce-back in a subject to provide an edema result.

### BACKGROUND

Edema shows observable swelling from fluid accumulation in body tissues of a person. Most commonly, edema occurs in feet, ankles, legs, and/or hands. The swelling is the result of the accumulation of excess fluid under the skin in the spaces within the tissues. According to one example, a pitting edema is demonstrated by applying pressure to a swollen area by depressing the skin with a finger. In fact, any form of pressure, such as from the elastic in socks, can induce pitting with this type of edema. Thus, symptoms of the pitting edema include swelling, which causes the skin surrounding it to tighten. The skin over the swollen area typically appears shiny and light, and, often, when a finger is placed on the swollen area an indentation is left on the skin.

Pitting edema is often diagnosed with a physical exam. For example, a doctor may apply pressure to the swollen skin for about 15 seconds to check for an indentation. After pressing the affected body part with a finger, the edema is classified based on the depth and duration of the indentation. The test provides, for example, a grade that ranges from Grade 1 to Grade 4. Grade 1 is associated with a pressure that typically leaves an indentation of 0-2 millimeters (“mm”) and rebounds generally immediately. This is the least severe type of pitting edema. Grade 2 is associated with a pressure that typically leaves an indentation of 3-4 mm and rebounds in fewer than 15 seconds. Grade 3 is associated with a pressure that typically leaves an indentation of 5-6 mm and takes up to 30 seconds to rebound. Grade 4 is associated with a pressure that typically leaves an indentation of 8 mm or deeper and takes more than 20 seconds to rebound.

Understanding the severity of edema is helpful in identifying the underlying cause and the best course of treatment. However, present testing methods are plagued by many problems. For example, some present testing methods are non-user friendly, relying mostly on those with vast medical experience (e.g., doctors) to accurately administer the test. Consequently, typical patients cannot properly and accurately administer self-tests, as they lack the proper knowledge of where to apply the pressure, how long to apply the pressure, and how deep to apply the pressure. In another example, some present testing methods lack consistency. Testing the wrong body part may result in inaccurate test results, leading the patient into the wrong treatment or, worse, into no treatment at all.

The present disclosure is directed to solving these and other problems, including problems associated with present edema testing.

### SUMMARY

According to some implementations of the present disclosure, a system includes an electronic interface that is configured to receive user input and to provide user instructions. The user input includes a user request for an edema test, and the user instructions include procedural steps for the edema test. The system further includes one or more sensors for detecting a change in skin characteristics of a subject, and a memory storing machine-readable instructions. The system further includes a control system having one or more processors configured to execute the machine-readable instructions. The instructions include the activation of the edema test automatically or in response to receiving the user request via the electronic interface. Upon activation, providing the user instructions for locating, via at least one of the one or more sensors, a skin area in the subject for testing. The instructions further include instructing a user to depress the skin area for causing a temporary indentation, and capturing, via at least one of the one or more sensors, one or more images of the temporary indentation over a period of time following the depression of the skin area by the user. The instructions also include analyzing the one or more images for characteristics of skin bounce-back, the skin bounce-back representing rebounding of the skin area after the temporary indentation. The instructions also include determining an edema result based on the characteristics of the skin bounce-back.

According to some other implementations of the present disclosure, a method includes activating an edema test via an electronic device, in response to receiving a user request. Upon activating the edema test, the method further includes locating for a user a skin area of a subject for testing of edema. In response to locating the skin area, the user is instructed to depress the skin area for causing a temporary indentation. One or more images of the temporary indentation are captured during an elapsed time period in which the skin area bounces back to a full or partial undepressed state. The one or more images are analyzed for characteristics of skin bounce-back. An edema result is determined based on the characteristics of the skin bounce-back.

According to yet other implementations of the present disclosure, a system is directed to determining an edema result. The system includes a probe having a proximal end and a distal end. The distal end has a distal surface for causing a temporary indentation in a skin surface of a subject. The system further includes an electronic device with a housing for enclosing internal components. The housing has an external surface on which the proximal end of the probe is removably attached. The electronic device further has an electronic interface configured to receive user input from a user and to provide user instructions. The user input includes a user request for an edema test. The user instructions include procedural steps for the edema test. The electronic device also has a camera for detecting a change in skin characteristics of the subject, and a memory storing machine-readable instructions. The electronic device also has a control system including one or more processors configured to execute the machine-readable instructions. The machine-readable instructions include to activate the edema test in response to receiving the user request via the electronic interface. Upon activation of the edema test, the user instructions are provided for locating via the camera the skin area in the subject for testing. The user is instructed to depress the skin area for causing the temporary indentation. One or more images of the temporary indentation are captured, via the camera, as the skin area bounces back to a full or partial undepressed state. The images are analyzed for characteristics of skin bounce-back. An edema result is determined based on the characteristics of the skin bounce-back.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of exemplary embodiments together with reference to the accompanying drawings. These drawings depict only exemplary embodiments, and are therefore not to be considered as limitations on the scope of the various embodiments or claims.

FIG. 1 is a functional block diagram of a system for determining an edema result, according to some implementations of the present disclosure.

FIG. 2 is a perspective view illustrating a mobile phone configured for an edema test in a subject, according to some implementations of the present disclosure.

FIG. 3 is a perspective view of the mobile phone of FIG. 2 prior to activation of the edema test, according to some implementations of the present disclosure.

FIG. 4 is a screenshot displayed on the mobile phone of FIG. 2 illustrating the activation of the edema test, according to some implementations of the present disclosure.

FIG. 5 is a screenshot displayed on the mobile phone of FIG. 2 illustrating locating skin areas for the edema test, according to some implementations of the present disclosure.

FIG. 6 is a screenshot displayed on the mobile phone of FIG. 2 illustrating a recommendation for test location, according to some implementations of the present disclosure.

FIG. 7 is a screenshot displayed on the mobile phone of FIG. 2 illustrating instructions for depressing the skin area in the test location, according to some implementations of the present disclosure.

FIG. 8 is a screenshot displayed on the mobile phone of FIG. 2 illustrating instructions for capturing one or more images of a temporary indentation, according to some implementations of the present disclosure.

FIG. 9 is a screenshot displayed on the mobile phone of FIG. 2 showing a plurality of captured images of the test location, according to some implementations of the present disclosure.

FIG. 10 is a screenshot displayed on the mobile phone of FIG. 2 showing an analysis of the captured images, according to some implementations of the present disclosure.

FIG. 11 is a screenshot displayed on the mobile phone of FIG. 2 showing a result for the edema test, according to some implementations of the present disclosure.

FIG. 12 is a screenshot displayed on the mobile phone of FIG. 2 showing a recommendation based on the result of the edema test, according to some implementations of the present disclosure.

FIG. 13 is a perspective view of a mobile phone with a probe assembly having a fixed end, according to some implementations of the present disclosure.

FIG. 14 is an exploded view of the probe assembly illustrated in FIG. 13, according to some implementations of the present disclosure.

FIG. 15 is a perspective view illustrating an edema test prior to causing a temporary indentation, using the fixed probe assembly illustrated in FIG. 13, according to some implementations of the present disclosure.

FIG. 16 is a perspective view illustrating the edema test of FIG. 15 during the causation of the temporary indentation, according to some implementations of the present disclosure.

FIG. 17 is a perspective view of a mobile phone with a probe having a movable end, according to some implementations of the present disclosure.

FIG. 18 is a cross-sectional view illustrating an undepressed state of the probe of FIG. 17, according to some implementations of the present disclosure.

FIG. 19 is a cross-sectional view illustrating a depressed state of the probe of FIG. 17, according to some implementations of the present disclosure.

FIG. 20 is a perspective view illustrating a probe end with protrusions, according to some implementations of the present disclosure.

FIG. 21 is a perspective view illustrating a probe with a fisheye lens having multiple filters, according to some implementations of the present disclosure.

FIG. 22 is a perspective view illustrating a probe with a spherical, flattened end, according to some implementations of the present disclosure.

FIG. 23 is a process flow diagram for an edema test, according to some implementations of the present disclosure.

While the invention is susceptible to various modifications and alternative forms, specific implementations have been shown by way of example in the drawings and will be described in further detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Various embodiments are described with reference to the attached figures, where like reference numerals are used throughout the figures to designate similar or equivalent elements. The figures are not drawn to scale and are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The various embodiments are not limited by the illustrated ordering of acts or events, as some acts may occur in different orders and/or concurrently with other acts or events. Furthermore, not all illustrated acts or events are required to implement a methodology in accordance with the present invention.

Elements and limitations that are disclosed, for example, in the Abstract, Summary, and Detailed Description sections, but not explicitly set forth in the claims, should not be incorporated into the claims, singly, or collectively, by implication, inference, or otherwise. For purposes of the present detailed description, unless specifically disclaimed, the singular includes the plural and vice versa. The word “including” means “including without limitation.” Moreover, words of approximation, such as “about,” “almost,” “substantially,” “approximately,” “generally,” and the like, can be used herein to mean “at,” “near,” or “nearly at,” or “within 3-5% of,” or “within acceptable manufacturing tolerances,” or any logical combination thereof, for example.

Generally, the present disclosure describes a method and system for detecting edema in a patient (also referred to herein as a subject), based on analyzing bounce-back (or rebounding) of skin when depressed. The bounce-back is analyzed, for example, to determine how long it takes for the depressed skin to fully or partially rebound. The bounce-back is optionally analyzed by determining a color change of the affected skin area over time. For example, a color change is used to provide additional information related to skin characteristics during the bounce-back. The additional information includes, for example, where an indentation has occurred, blood flow at an indentation site, edema fluid flow at the indentation site, etc. Optionally, the system includes a probe that attached to an electronic device, such as a mobile device, to aid in applying pressure to the skin in a repeatable manner. The probe further allows a camera to view the test area through a lens or window of the probe 100% of the time while performing the edema test.

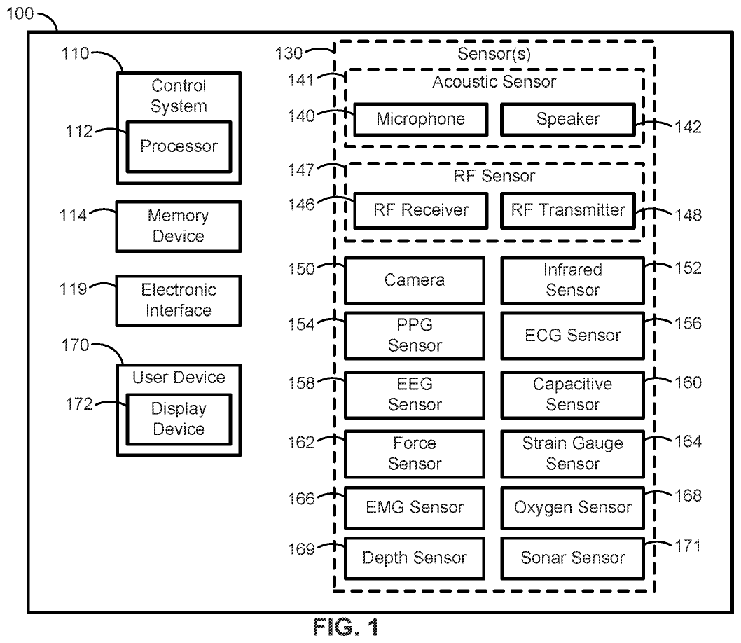

Referring to FIG. 1, a system 100, according to some implementations of the present disclosure, is illustrated. The system 100 includes a control system 110, a memory device 114, an electronic interface 119, one or more sensors 130, and one or more user devices 170. According to one exemplary implementation, discussed in more detail below, the system 100 includes a user mobile device, such as a mobile phone. The user device 170, such as the mobile phone, optionally includes in one exemplary implementation the control system 110, the memory device 114, the electronic interface 119, and at least one sensor 130, as further described in more detail below.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the user device 170, within a housing of one or more of the sensors 130, or any combination thereof. Like the control system 110, the memory device 114 can be centralized (within one such housing) or decentralized (within two or more of such housings, which are physically distinct).

The electronic interface 119 is configured to receive data (e.g., physiological data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

The one or more sensors 130 of the system 100 include a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, a depth sensor 169, a sonar sensor 171, or any combination thereof. Generally, each of the one or more sensors 130 is configured to output sensor data that is received and stored in the memory device 114 or one or more other memory devices.

While the one or more sensors 130 are shown and described as including each of the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, a depth sensor 169, and a sonar sensor 171, the one or more sensors 130 can include any combination and any number of each of the sensors described and/or shown herein. The physiological data generated by one or more of the sensors 130 can be used by the control system 110 to determine an edema result based on characteristics of skin bounce-back, as described in more detail below.

The microphone 140 outputs sound data that can be stored in the memory device 114 and/or analyzed by the processor 112 of the control system 110. For example, the microphone 140 can be used to record sound during an edema test session. The microphone 140 can be coupled to or integrated in the user device 170.

The speaker 142 outputs sound waves that are audible to a user of the system 100. The speaker 142 can be used, for example, as an alarm clock or to play an alert or message to the user (e.g., in response to an event). The speaker 142 can be coupled to or integrated in the external device 170.

The microphone 140 and the speaker 142 can be used as separate devices. In some implementations, the microphone 140 and the speaker 142 can be combined into an acoustic sensor 141 (e.g., a sonar sensor), as described in, for example, International (PCT) Patent Publication Nos. WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz). Based at least in part on the data from the microphone 140 and/or the speaker 142, the control system 110 can determine a location of the subject or subject's body part to be tested, and/or one or more characteristics of the skin bounce-back that is described in more detail below. Optionally or alternatively, the control system 110 determines a distance to a body part of the subject, mapping of the indentation, and/or respiration of the subject. According to another exemplary implementation, the control system 110 determines a velocity through a Doppler shift, which allows the control system 110 to know if the system 100 is not being held still by the subject or the user. Alternatively, the velocity is determined using an accelerometer. Optionally, if a determination is made that the system 100 is not being held still, a notification is provided to request that the subject or the user should “hold the device still.” In addition or alternatively, the control system 110 can determine sleep-related parameters described in herein such as, for example, a respiration signal, a respiration rate, an inspiration amplitude, an expiration amplitude, an inspiration-expiration ratio, a number of events (e.g., snoring, an apnea, a hypopnea, a restless leg, a sleeping disorder, choking, an increased heart rate, labored breathing, an asthma attack, an epileptic episode, a seizure, or any combination thereof) per hour, a pattern of events, a sleep state, a sleep stage, pressure settings of the respiratory device 122, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. Methods for determining sleep states and/or sleep stages from physiological data generated by one or more of the sensors, such as sensors 130, are described in, for example, WO 2014/047310, US 2014/0088373, WO 2017/132726, WO 2019/122413, and WO 2019/122414, each of which is hereby incorporated by reference herein in its entirety.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and these data can be analyzed by the control system 110 to determine a location of the subject or the subject's body part to be tested, and/or one or more of the skin bounce-back characteristics described herein, the distance to the body part of a subject, mapping of the indentation, respiration of the subject, and/or the velocity of the system 100. An RF receiver (either the RF receiver 146 and the RF transmitter 148 or another RF pair) can also be used for wireless communication between the control system 110, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a radar sensor). In such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the skin bounce-back characteristics.

The infrared (IR) sensor 152 outputs infrared image data reproducible as one or more infrared images (e.g., still images, video images, or both) that can be stored in the memory device 114. The IR sensor 152 can be used in conjunction with the camera 150 when measuring the presence, location, and/or movement of a depressed skin area described herein. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more user characteristics, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. According to an exemplary embodiment, the PPG sensor 154 is part of the mobile phone, such as being part of a camera. According to another exemplary embodiment, the PPG sensor 154 is part of the camera and the flash of the mobile phone. The PPG sensor 154 detects a change in intensity of wavelengths, e.g., red, infrared, or green (which are the most common wavelengths). The detected change in wavelength intensity is provided as data for determining an edema result. The PPG sensor 154 can alternatively or additionally be worn by the user, can be embedded in clothing and/or fabric that is worn by the user, can be embedded in and/or coupled to its associated headgear (e.g., straps, etc.), etc. The physiological data of the PPG sensor 154 is optionally used to measure a perfusion index, which is correlated with an analysis of one or more images for confirming an edema result (disclosed in more detail below).

The ECG sensor 156 outputs physiological data associated with electrical activity of the heart of the user. According to exemplary embodiments, the ECG sensor 156 is worn by the user (e.g., in a smart watch), is embedded in clothing and/or fabric that is worn by the user, is embedded in and/or coupled to its associated chest band (e.g., straps, etc.), etc. The EEG sensor 158 outputs physiological data associated with electrical activity of the brain of the user. According to exemplary embodiments, the EEG sensor 158 is worn by the user, is embedded in clothing and/or fabric that is worn by the user, is embedded in and/or coupled to its associated headgear (e.g., straps, etc.), etc.

The capacitive sensor 160, the force sensor 162, and the strain gauge sensor 164 output data that can be stored in the memory device 114 and used by the control system 110 to determine one or more of the skin bounce-back characteristics described herein. The EMG sensor 166 outputs physiological data associated with electrical activity produced by one or more muscles. The oxygen sensor 168 outputs oxygen data indicative of an oxygen concentration of blood. The oxygen sensor 168 can be, for example, an ultrasonic oxygen sensor, an electrical oxygen sensor, a chemical oxygen sensor, an optical oxygen sensor (such as a PPG sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The depth sensor 169 detects a distance between the sensor and a patient or user, or a number of distances (or depths) to provide a three-dimensional (3D) representation of the skin area in which the temporary indentation is formed. For example, in reference to testing for edema (as disclosed in more detail below), the depth sensor 169 detects a change in depth caused by a temporary indentation in a skin surface. The sonar sensor 171 also detects the change in depth using acoustic measurement.

While shown separately in FIG. 1, any combination of the one or more sensors 130 can be integrated in and/or coupled to any one or more of the components of the system 100, including the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, at least one of the one or more sensors 130 is not coupled to the control system 110 or the user device 170. Sensor data may be transferred to the control system 110 from the uncoupled sensor 130 via indirect means of communication, e.g., via a flash drive, an external hard drive, etc.

The user device 170 includes a display device 172. The user device 170 can be, for example, a mobile device such as a smart phone, a tablet, a laptop, or the like. Alternatively, the user device 170 can be an external display system, an external user input system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

While the control system 110 and the memory device 114 are described and shown in FIG. 1 as being a separate and distinct component of the system 100, in some implementations, the control system 110 and/or the memory device 114 are integrated in the user device 170. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the user according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. Thus, various systems can be formed using any portion or portions of the components shown and described herein and/or in combination with one or more other components.

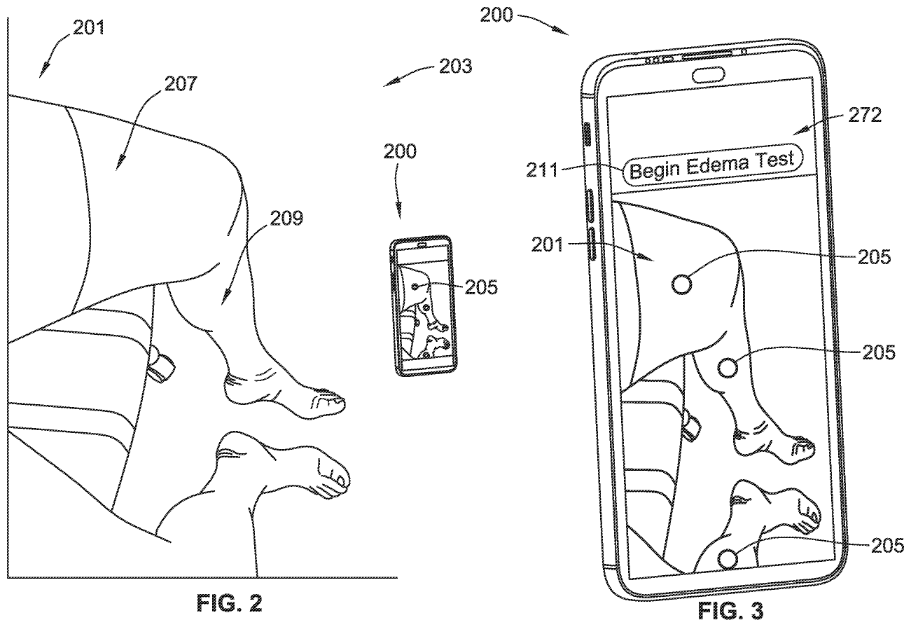

Referring to FIG. 2, the system disclosed above is illustrated by way of example in the form of a mobile phone 200, but other camera-enabled electronic devices may also be used. One or more of the electronic devices have a control system including one or more processors configured to execute machine-readable instructions, such as upon activating the edema test, locating for a user a skin area of a subject for testing of edema; in response to locating the skin area, instructing the user to depress the skin area for causing a temporary indentation; capturing one or more images of the temporary indentation during an elapsed time period in which the skin area bounces back to a full or partial undepressed state; analyzing the one or more images for characteristics of skin bounce-back; determining an edema result based on the characteristics of the skin bounce-back; etc.

The mobile phone 200 is configured to initiate an edema test in a subject 201, which in this example is a patient in a home environment 203. The mobile phone 200 facilitates in a user-friendly, simple manner the taking of the edema test in one or more skin areas 205 of the patient 201. Examples of the skin areas 205 for the edema test are located in a thigh area 207 or a calf area 209 of either leg of the patient 201. According to other examples, the skin areas 205 are located on an arm or a foot of the patient 201. The edema test, as disclosed in more detail below, is administered by the patient 201 or by a third-party, such as a family member or a medical professional (e.g., a doctor, a nurse, a trained specialist, etc.). Although in this example the edema test is described in a home environment 203, according to optional or alternative examples the edema test is administered in others environments, such as a medical facility (e.g., a hospital, a doctor's office, etc.).

Referring to FIG. 3, the mobile phone 200 has a display device 272 via which the patient 201 (or a third-party user, such as a medical doctor, nurse, or caregiver) interacts for performing the edema test. The display device 272 guides the patient 201 through the steps of the edema test, which may include starting with an activation step illustrated via an activation icon 211. Optionally, the display device 272 shows an initial augmented reality (AR) screenshot in which the skin areas 205 are identified for the patient 201 as potential test locations.

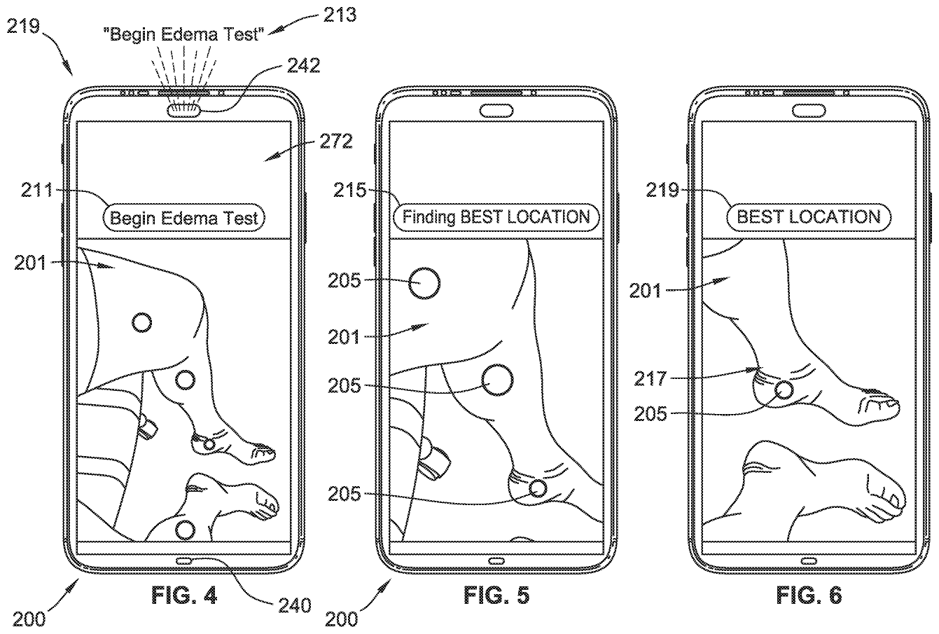

Referring generally to FIGS. 4-12, the edema test will be described in more detail. The edema test includes user input in which a user request is received for activating the edema test, and user instructions for guiding the user through the procedural steps of the edema test.

Referring specifically to FIG. 4, the edema test is activated by pressing the activation icon 211. In an alternative implementation, the edema test may automatically be activated by, for example, accessing or opening the test, such as on the mobile phone 200, and the user request therefore comprises the user accessing or opening the test. The activation step is performed in response to receiving a user request via the display device 272, which here operates as at least a portion of an electronic interface 219. The display device 272 of the mobile phone 200 functions in accordance with the display device 172 illustrated in FIG. 1, and the electronic interface 219 of the mobile phone 200 functions in accordance with the electronic interface 119 illustrated in FIG. 1. In this example, the user request is received by pressing the activation icon 211 on the display device 272.

Optionally, in addition or alternative to the pressing of the activation icon 211, an audible command is received in a microphone 240 from the patient 201 (or other third-party). Optionally yet, the activation of the edema step is confirmed with an audio message 213 emitted by the mobile phone 200 via a speaker 242. The microphone 240 of the mobile phone 200 functions in accordance with the microphone 140 illustrated in FIG. 1, and the speaker 242 of the mobile phone 200 functions in accordance with the speaker 142 illustrated in FIG. 1.

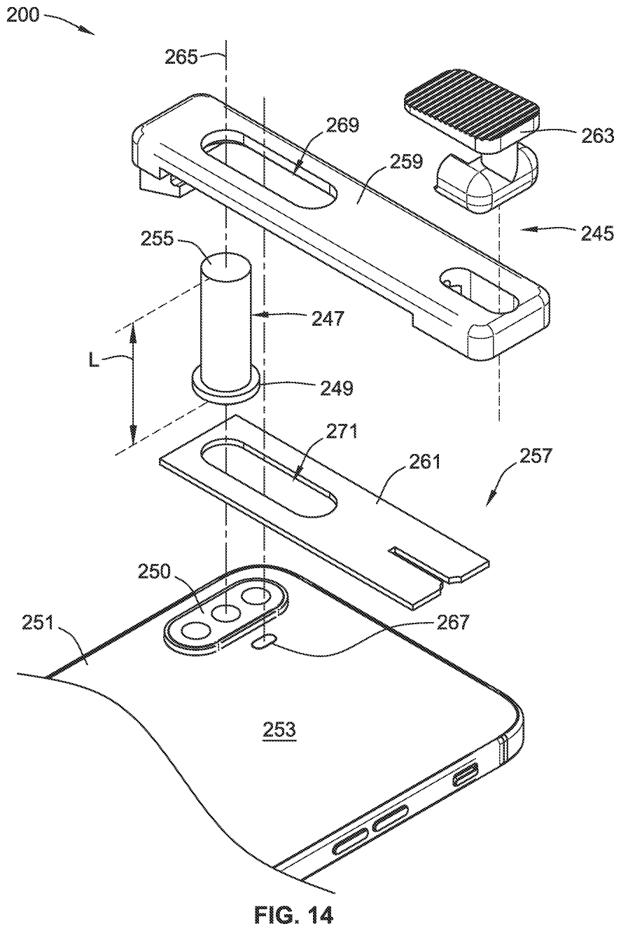

Referring specifically to FIG. 5, upon activation of the edema test, the edema test procedure provides user instructions in which one or more of potential skin areas 205 are located in the patient 201 for testing. The mobile phone 200 scans via one or more of its sensors the patient 201 to find potential targets for the test in body of the patient 201. The one or more sensors include one or more of the sensors described above in reference to FIG. 1, including, for example, a camera 250 (illustrated in FIG. 14) that functions in accordance with the camera 150 illustrated in FIG. 1. While locating the test area, the edema test optionally shows a finding (or location) icon 215 on the display device 272. For clarity, it is noted that the potential skin areas 205 are artificially superimposed as augmented reality (AR) icons over a live display of the body of the patient 201. Thus, the circular icons displayed in FIG. 5 (referenced as skin areas 205) are only viewable on the mobile phone 200, superimposed over the physical, live display of the body of the patient 201.

Referring specifically to FIG. 6, after scanning the patient 201 and locating one or more potential test area targets, one of the located skin areas 205 is selected for the edema test. The selected skin area 205 is automatically selected in response to the scanning of the patient 201, or is manually selected by the patient 201 (or other third-party). In the illustrated example, a determination is made that the skin area 205 in a heel portion 217 of the foot of the patient 201 is the best location. Optionally, a “best location” icon 219 is presented on the display device 272.

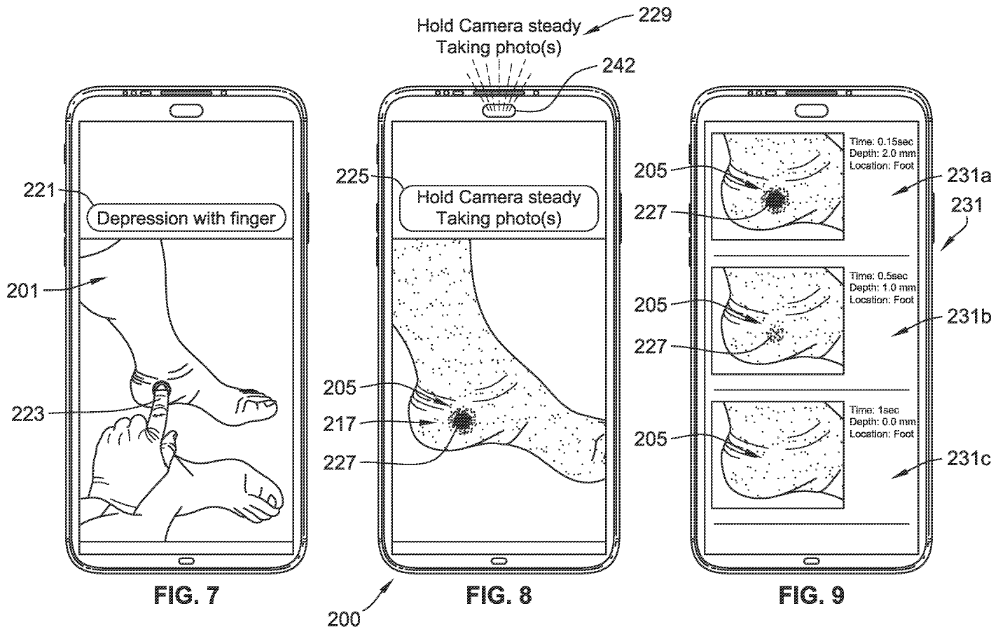

Referring specifically to FIG. 7, after locating the skin area 205 for the edema test, a depression icon 221 is provided that instructs the patient 201 (or other third-party user) to depress the located skin area 205 for causing a temporary indentation 227 (shown in FIG. 8) in the skin of the patient 201. The depression, according to this example, is optionally caused by a finger 223 pressed against the skin area 205. Alternatively, as described in more detail below, a physical probe is used for forming the temporary indentation 227 (shown in FIG. 8).

Referring specifically to FIG. 8, after removing the finger 223 (shown in FIG. 7) from the skin area 205, an image capturing icon 225 indicates that one or more images or being captured via at least one of the sensors of the mobile phone 200 (e.g., the camera 250 illustrated in FIG. 14). As illustrated in this exemplary embodiment, the image capturing icon 225 provides a request to “Hold Camera steady” because the mobile phone 200 is “Taking photo(s)” of the temporary indentation 227 caused in the heel portion 217. Optionally or alternatively, a similar or identical audio indication 229 is provided via the speaker 242. The images capture in real time the change in depth of the temporary indentation 227 as the skin area 205 rebounds.

Referring specifically to FIG. 9, the edema test captures a plurality of images 231, which include at least three images 231a-231c showing rebounding of the skin area 205 over a period of time following the depression of the skin area 205. The rebounding, depending on severity and/or presence of edema, manifests itself via certain characteristic of skin bounce-back after causing the temporary indentation 227. The characteristics are dependent on the elapsed time after the causation of the temporary indentation 227, and include, for example, depth and/or color of the temporary indentation 227 at a specific time interval.

According to one implementation, the images 231 are captured during a predetermined period of time, e.g., 60 seconds. According to another implementation, the images 231 are captured until the depth of the temporary indentation 227 is back to the original undepressed state of 0.0 mm. According to yet another implementation, the images 231 are captured until a predetermined depth is achieved, which is not necessarily the depth of 0.0 mm. The predetermined depth may be determined based on, for example, an initial depth of the temporary indentation 227 caused by the user (e.g., using a finger 223 or probe 247 as described herein) and from which initial depth the skin bounces back (once the finger 223 or probe 247 is removed from the skin area 205).

According to a first image 231a, at a time interval 0.15 seconds after causing the temporary indentation 227, the depth of the temporary indentation 227 is 2.0 mm and shows a first discoloration. Thus, this first image 231a captures skin bounce-back between the temporary indentation 227 and a partial undepressed state, which represents a partial rebounding of the skin area 205.

According to a second image 231b, at a time interval 0.5 seconds after causing the temporary indentation 227, the depth of the temporary indentation 227 is 1.0 mm and shows a second discoloration. The second discoloration is closer to a normal skin color than the first discoloration. Thus, this second image 231b captures a subsequent elapsed period of time between the temporary indentation 227 and a subsequent partial undepressed state. The subsequent elapsed period of time of 0.5 seconds is longer than the initial elapsed period of time of 0.15 seconds. The subsequent partial undepressed state in which the depth of the temporary indentation 227 is 1.0 mm represents a subsequent partial rebounding of the skin area 205. The subsequent partial rebounding of 1.0 mm is closer to the original undepressed state of 0.0 mm than the partial undepressed state of 2.0 mm.

According to a third image 231c, at a time interval of one second after causing the temporary indentation 227, the skin bounce-back is now back to normal with a 0.0 mm depth and no discoloration. Thus, the total time for achieving full or complete bounce-back of the skin was one second.

In addition to images captured after the causation of the temporary indentation 227, one or more images are optionally captured before and/or during the depression of the skin area 205. These additional images are useful in providing a comparison between a depressed and an undepressed state of the skin area 205.

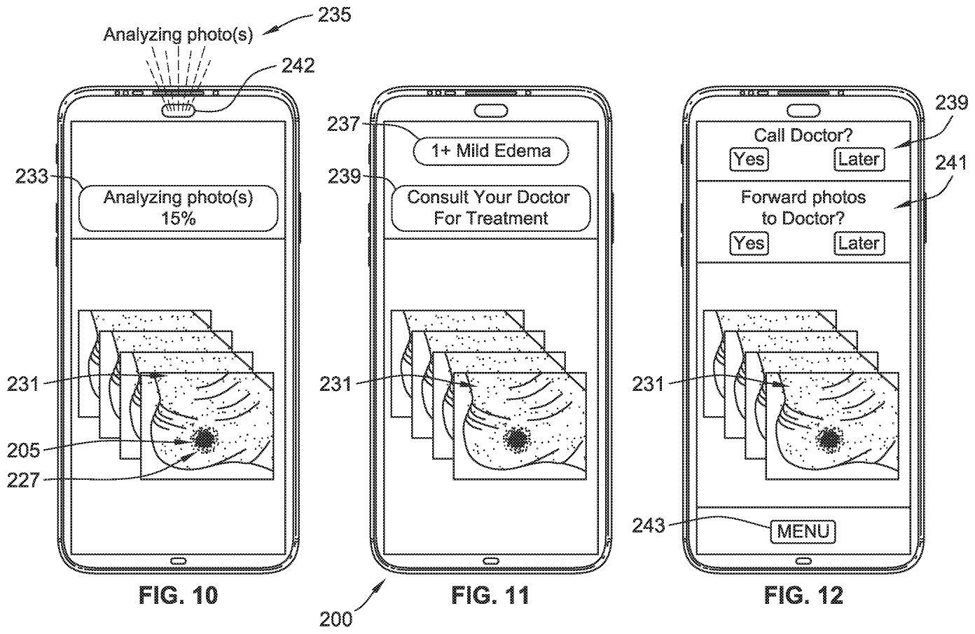

Referring specifically to FIG. 10, the captured images 231 are analyzed to determine characteristics of the skin bounce-back, which represents the rebounding of the skin area 215 after the temporary indentation 227. As illustrated in this exemplary embodiment, an optional analysis icon 233 provides an indication that the test is “Analyzing photo(s)” and is currently at “15%” of the analysis. Optionally or alternatively, a similar or identical audio indication 235 is provided via the speaker 242.

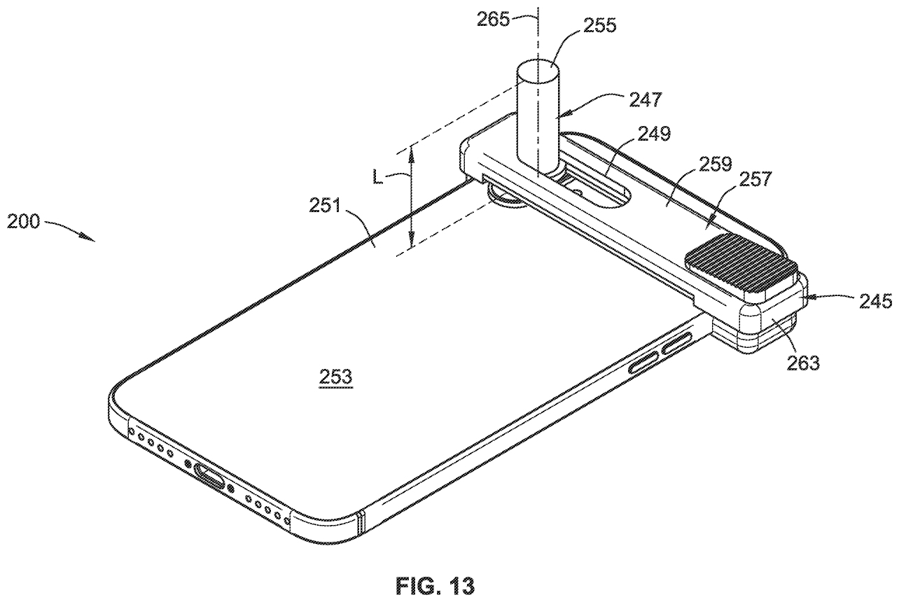

The analyzed characteristics optionally include a compensation for an elapsed time during which the skin area 205 was obstructed from view, e.g., by the finger 223 (shown in FIG. 7) or by a probe 247 (shown in FIG. 13). The compensation optionally accounts, for example, an amount of time during which the finger 223 obstructed the temporary indentation 227 and the respective rebounding of the skin that has occurred during that amount of time. The compensation is automatically determined, using live imaging, or is predetermined based on general statistics and/or assumptions. For example, the compensation is based on the assumption that an average person obstructs the temporary indentation 227 for 0.5 seconds, during which the skin generally rebounds 1 mm.

Referring specifically to FIG. 11, an edema result is displayed based on characteristics of the skin bounce-back analyzed in the captured images 231 of the mobile phone 200. The edema result, for example, displays an edema score 237 that indicates a case of “1+ Mild Edema.” The edema result optionally provides a test follow-up recommendation 239, which in this example recommends that the patient consult a doctor for treatment or change a medication. Although not illustrated, the edema result is optionally provided in audio form, similar to the audio indication 235 disclosed above in reference to FIG. 10. It is further understood that test indications, including test instructions and results, are provided in visual and/or audio form in accordance with the above disclosure.

As already mentioned above, the edema result is optionally confirmed using physiological data of the patient detected via a PPG sensor. The physiological data are used to measure the perfusion index and, then, the perfusion index is correlated with the edema result. According to an alternative implementation, the characteristics of the skin bounce-back are further selected from a group consisting of oxygen data (e.g., perfusion index, SpO₂, etc.) and flushness data. Thus, the edema result is optionally based one or more of bounce-back characteristics selected from PPG data, hemoglobin data, oxygen data, flushness data, and bounce-back data.

Referring specifically to FIG. 12, further supplemental test follow-up options are presented. For example, after determining the edema result, a call option 239 is displayed to call a doctor, and a forwarding option 241 is displayed to send the captured images 231 to the doctor. The test follow-up options are manually accepted or are automatically implemented. For example, depending on the severity of the edema result, a phone call is automatically initiated or scheduled with a medical facility.

A menu icon 243 facilitates administering one or more subsequent edema tests and/or other options related to the present edema test. Optionally, the subsequent edema tests locate the same skin area for testing as the present edema test. The same skin area increases consistency and/or reliability of the test results.

Referring to FIGS. 13 and 14, the mobile phone 200 is illustrated with an attachment that is in the form of a probe assembly 245, for causing the temporary indentation 227 illustrated in FIG. 7. Thus, according to this example, instead of using the finger 223 (as shown in FIG. 7), the edema test uses the probe assembly 245.

The probe assembly 245 includes the probe 247 that has a proximal end 249 reversibly attached to an exterior surface 251 of a housing 253 of the mobile phone 200. The probe 247 further has a distal end 255 that is used to cause the temporary indentation 227 (shown in FIG. 7). The distal end 255 is fixed relative to the proximal end 249.

In addition to the probe 247, the probe assembly 245 includes an attachment assembly 257 that reversibly fixes the proximal end 249 to the exterior surface 251. The attachment assembly 257 includes an attachment base 259 mounted over an attachment plate 261 (shown in FIG. 14) and fixed to the mobile phone 200 via an attachment latch 263.

Referring specifically to FIG. 14, the probe 247 is substantially cylindrical in shape and has an imaging conduit 265 that is at least in part transparent or translucent. The imaging conduit 265 extends through an interior portion of the probe 247 throughout a length L. The imaging conduit 265 is in visual communication with the camera 250 (shown in FIG. 14) of the mobile phone 200, generally along a line of sight depicted by the imaging conduit 265.

The attachment assembly 257 provides a clearance for an illumination element 267 of the mobile phone 200, which is typically in the form of a flash for the camera 250. The clearance is achieved by a top slotted hole 269 of the attachment base 259 overlapping a bottom slotted hole 271 of the attachment plate 261. Illumination from the flash 267 optionally illuminates a body part of the patient 201 during the edema test disclosed above. For example, the flash 267 illuminates skin areas 205 (shown in FIG. 5) when locating the test area.

Optionally, the probe 247 is formed at least in part from a translucent and/or transparent material, which is the same as or similar to the imaging conduit 265. For example, walls of the probe 247, which extend between the proximal end 249 and the distal end 255), are made from a translucent material and/or a transparent material. The translucent and/or transparent characteristic of the probe 247 advantageously allows illumination of a subject's skin, using ambient light, without requiring a dedicated light source, such as the illumination element 267 of the mobile phone 200.

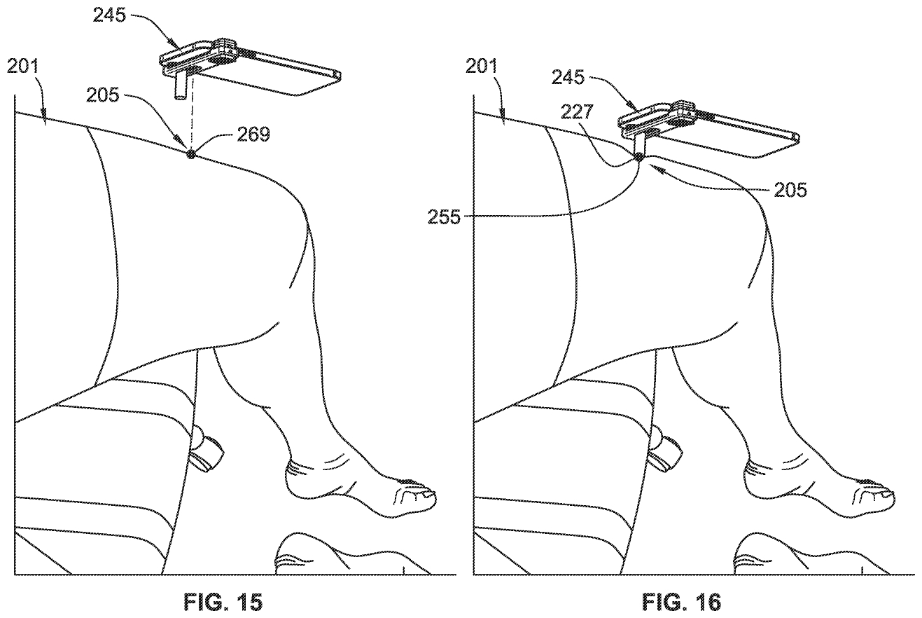

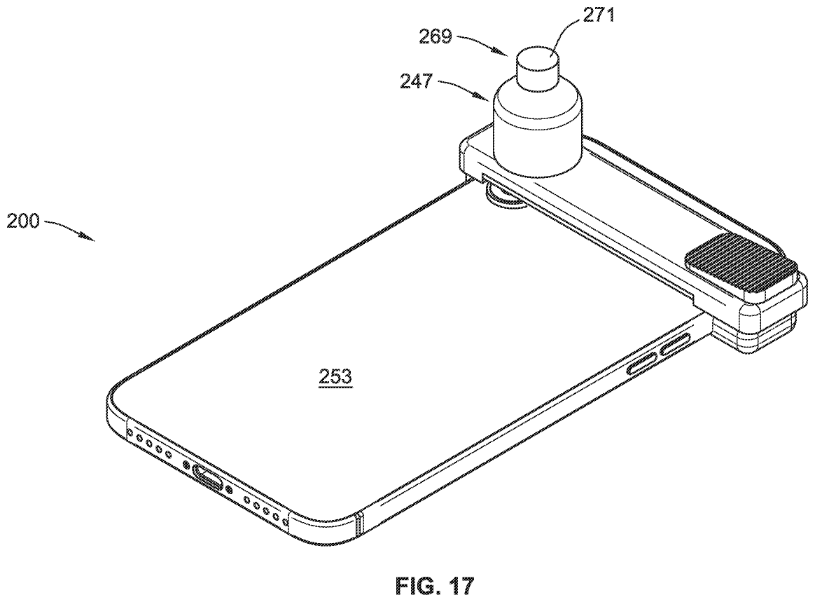

Referring to FIGS. 15 and 16, the probe assembly 245 is used by to cause the temporary indentation 227 (shown in FIG. 16). Thus, as already disclosed above, the temporary indentation 227 is achieved in various ways, including using the finger 223 (as shown in FIG. 7) and the probe assembly 245. According to this example, the distal end 255 makes contact with the skin area 205 and causes the temporary indentation 227. As further illustrated in this example, an illumination dot 269 is formed by the flash 267 (illustrated in FIG. 14) to visually and precisely identify the skin area 205 for the user 201 on the body of the patient 201.

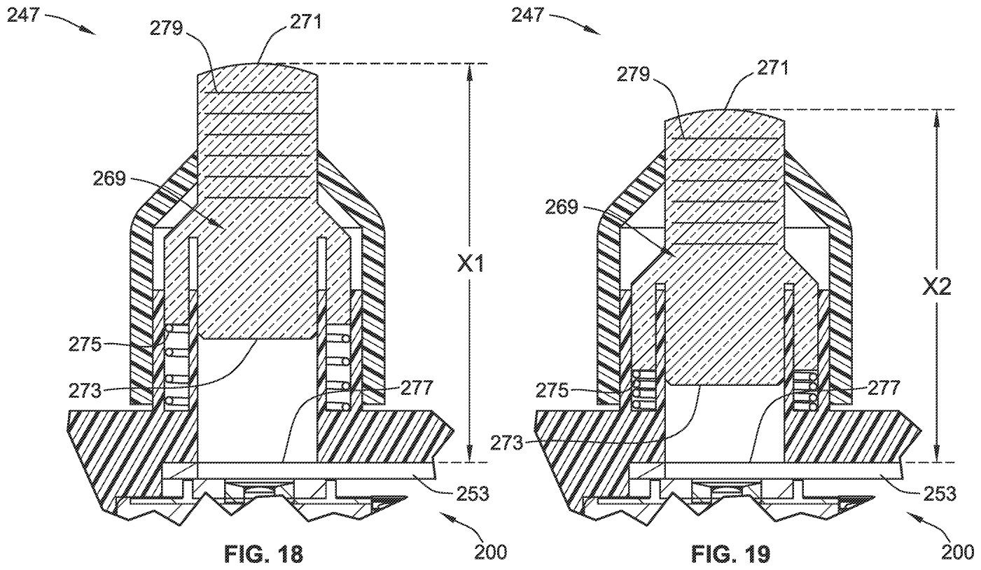

Referring generally to FIGS. 17-19, according to an alternative embodiment, the probe 247 has a movable element 269 having a movable distal end 271 for causing the temporary indentation 227 (shown in FIG. 8). The movable element 269 moves relative to the housing 253 of the mobile phone 200. A movable proximal end 273 (shown in FIGS. 18 and 19) of the probe 247 is closer to the housing 253 than the movable distal end 271.

Referring more specifically to FIGS. 18 and 19, the movable distal end 271 is supported by a biasing element 275. According one example, the biasing element 275 is a spring that extends between (a) a fully extended mode in an original undepressed mode (as illustrated in FIG. 18) and (b) a compressed mode when the temporary indentation 227 (shown in FIG. 8) is caused (as illustrated in FIG. 19). For example, in the fully extended mode the movable distal end 271 is at a first distance X1 from a top surface 277 of the housing 253. In the compressed mode, the movable distal end 271 is at a second distance X2 from the top surface 277. The first distance X1 is greater than the second distance X2.

To aid in measuring movement, internal marks 279 are provided internally within the movable element 269. As the movable element 269 is depressed, such as when pressing against a skin surface 205 (as illustrated in FIG. 16), the internal marks 279 provide a visual gauge for determining the depth of the caused temporary indentation 227 (also shown in in FIG. 16).

Referring generally to FIGS. 20-22, the probe 247 has various optional features near its distal end 255. According to the example illustrated in FIG. 20, a plurality of protrusions 281 that extend from an end surface 283 of the distal end 255. The protrusions 281 form a specific pattern when causing the temporary indentation 227 (illustrated in FIG. 16). Relative to a general flat surface, the protrusions 281 form an irregular surface for causing the temporary indentation.

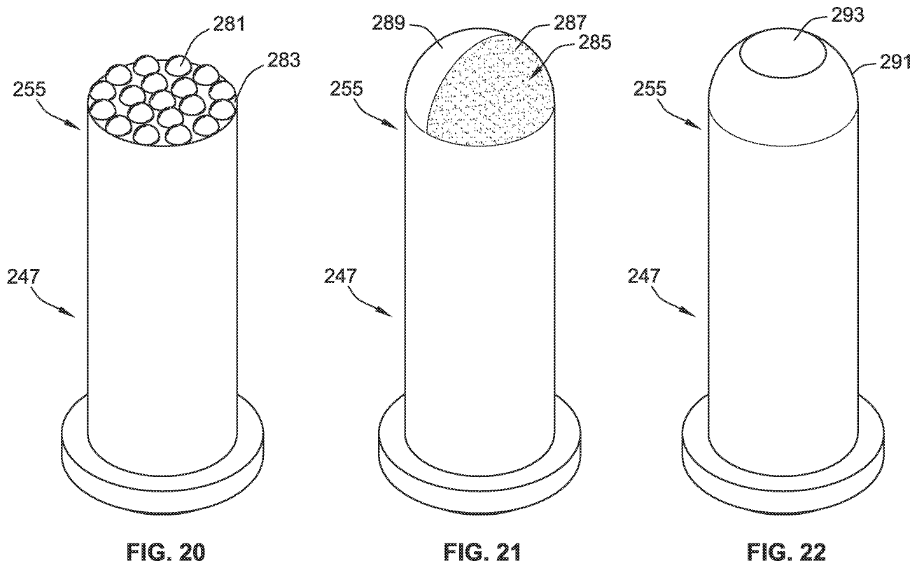

According to the example illustrated in FIG. 21, the distal end 255 includes a fisheye lens 285 for capturing wide-angle images. Optionally, the fisheye lens 285 includes at least one filter for providing various visualization of the captured images. For example, the fisheye lens 285 includes a first filter 287 for capturing an image of a first color and a second filter 289 for capturing an image of a second color. According to the example illustrated in FIG. 22, the distal end 255 has a half-sphere shape 291 with a flattened top portion 293. The flattened top portion 291 provides a precise, focused area of contact for causing temporary indentation 227 (illustrated in FIG. 16), while the half-sphere shape 291 provides a wider-angle for the captured image.

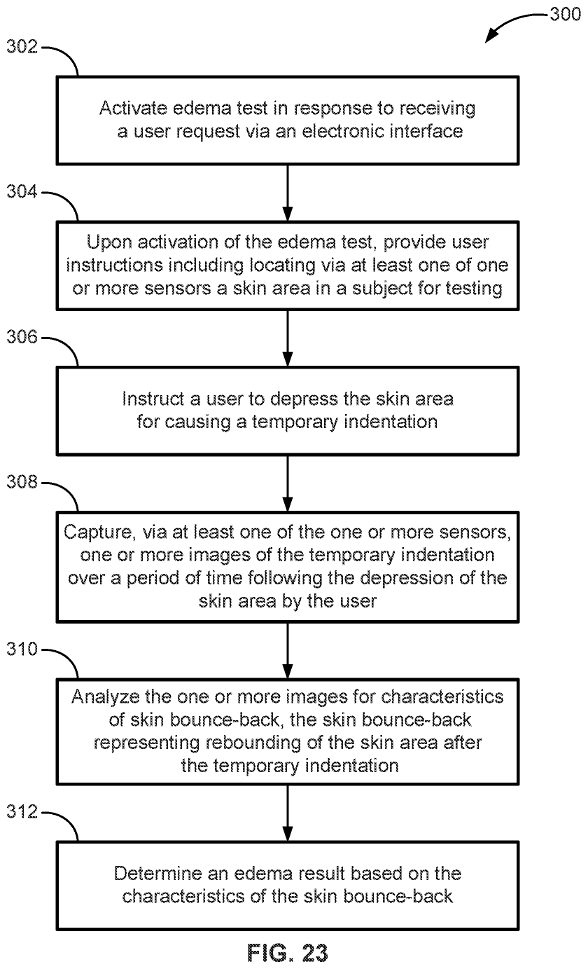

Referring to FIG. 23, an edema test procedure 300 is illustrated according to some implementations of the present disclosure. The edema test procedure can be implemented using any combination or aspects of the systems described herein. At step 302, the edema test is activated in response to receiving a user request via an electronic interface. At step 304, upon activation of the edema test, user instructions are provided. The user instructions include locating via at least one of one or more sensors a skin are in a subject for testing. At step 306, the user is instructed to depress the skin area for causing a temporary indentation. At step 308, one or more images are captured of the temporary indentation, via at least one of the one or more sensors. The images are captured over a period of time following the depression of the skin area by the user. At step 310, the images are analyzed for characteristics of skin bounce-back, which represents rebounding of the skin area after the temporary indentation. At step 312, an edema result is determined based on the characteristics of the skin bounce-back.

According to one or more illustrative embodiments, any of the systems or methods described above further detect or monitor edema in a subject in response to treatment of at least one edema-related condition. The edema-related condition is selected from a group consisting of one or more sleep-related conditions and/or respiratory-related conditions. For example, the detecting or monitoring of edema occurs before, during, or after the treatment of the sleep-related conditions and/or respiratory-related conditions. By way of further example, at least one of the conditions is one or more of a Disordered Breathing condition and a Chronic Obstructive Pulmonary Disease (COPD) condition.

According to one or more illustrative embodiments, any of the systems or methods described above further detect or monitor edema in a subject in response to treatment of lymphedema. For example, the detecting or monitoring of edema occurs before, during, or after the treatment of the lymphedema. The lymphedema includes primary lymphedema or secondary lymphedema. The primary lymphedema is caused by one or more of Milroy's disease, Meige disease, or Late-onset lymphedema. The secondary lymphedema is a result of a procedure or condition that results in damage or removal of lymph nodes or lymph vessels. For example, the secondary lymphedema is caused by one or more of a surgical operation, cancer, radiation treatment, or infection of the lymph nodes.

Devices, methods, and/or garments used in the treatment of lymphedema are described in, for example, PCT/US2019/055474 and WO2020/077008, each of which is hereby incorporated by reference herein in its entirety. For example, lymphedema treatments include compression therapy, which is optionally administered with a compression garment as described in PCT/US2019/055474 and WO2020/077008.

One or more elements or aspects or steps, or any portion(s) thereof, from one or more of any of claims 1-87 below can be combined with one or more elements or aspects or steps, or any portion(s) thereof, from one or more of any of the other claims 1-87 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A system comprising:
an electronic interface configured to receive user input and to provide user instructions, the user input including a user request for an edema test, the user instructions including procedural steps for the edema test;
one or more sensors for detecting a change in skin characteristics of a subject;
a memory storing machine-readable instructions; and
a control system including one or more processors configured to execute the machine-readable instructions to
activate the edema test automatically or in response to receiving the user request via the electronic interface,
upon activation of the edema test, provide the user instructions including locating via at least one of the one or more sensors a skin area in the subject for testing,
instruct a user to depress the skin area for causing a temporary indentation,
capture, via at least one of the one or more sensors, one or more images of the temporary indentation over a period of time following the depression of the skin area by the user,
analyze the one or more images for characteristics of skin bounce-back, the skin bounce-back representing rebounding of the skin area after the temporary indentation, and
determine an edema result based on the characteristics of the skin bounce-back.

2. The system of claim 1, further comprising a mobile device in which one or more of the electronic interface, the one or more sensors, the memory, and the control system are incorporated.

3. The system of claim 1, wherein at least one of the one or more sensors is a camera of a mobile phone.

4. The system of claim 1, wherein the temporary indentation is caused by a finger of the user.

5. The system of claim 1, to wherein the edema result includes an edema score.

6-7. (canceled)

8. The system of claim 1, wherein the characteristics of the skin bounce-back include (i) a length of time between causing the temporary indentation and achieving the original undepressed state, (ii) a skin color change during the skin bounce-back, or (iii) both (i) and (ii).

9. The system of claim 8, wherein the length of time is an initial elapsed period of time between the temporary indentation and a partial undepressed state, the partial undepressed state representing a partial rebounding of the skin area.

10. The system of claim 9, wherein the length of time is a subsequent elapsed period of time between the temporary indentation and a subsequent partial undepressed state, the subsequent elapsed period of time being longer than the initial elapsed period of time, the subsequent partial undepressed state representing a subsequent partial rebounding of the skin area that is closer to the original undepressed state than the partial undepressed state.

11. (canceled)

12. The system of claim 1, wherein analyzing the one or more images includes compensation for an elapsed time period during which the skin area is obstructed.

13-15. (canceled)

16. The system of claim 1, further comprising executing the machine-readable instructions to automatically implement a remedial action based on the edema result, wherein the remedial action includes automatically contacting a medical facility.

17-18. (canceled)

19. The system of claim 1, wherein a depth of the temporary indentation is determined in real time for achieving the edema result.

20. The system of claim 1, wherein at least one of the one or more sensors is a photo-plethysmography (PPG) sensor, the one or more processors being further configured to execute the machine-readable instructions to
measure a perfusion index; and
correlate the perfusion index with the analysis of the one or more images to confirm the edema result.

21-22. (canceled)

23. The system of claim 1, wherein the at least one of the one or more sensors is selected from a group consisting of a camera, a depth sensor, and a sonar sensor.

24. The system of claim 1, further comprising:
a housing in which are incorporated within the electronic interface, one or more sensors, the memory, and the control system; and
a probe having proximal end attached to an exterior surface of the housing, the probe having a distal end that is used by the user to cause the temporary indentation.

25. The system of claim 24, wherein the probe is substantially cylindrical in shape.

26-28. (canceled)

29. The system of claim 24, wherein the probe includes a lens at the distal end, the lens being in visual communication with the at least one of the one or more sensors.

30-39. (canceled)

40. The system of claim 1, further comprising:
a housing in which are incorporated within the electronic interface, one or more sensors, the memory, and the control system; and
a probe having proximal end attached to an exterior surface of the housing, the probe having a movable distal end that is used by the user to cause the temporary indentation.

41. The system of claim 40, wherein the probe has internal marks for measuring a depth of the temporary indentation.

42-52. (canceled)

53. A method comprising:
activating an edema test via an electronic device;
upon activating the edema test, locating for a user a skin area of a subject for testing of edema;
in response to locating the skin area, instructing the user to depress the skin area for causing a temporary indentation;
capturing one or more images of the temporary indentation during an elapsed time period in which the skin area bounces back to a full or partial undepressed state;
analyzing the one or more images for characteristics of skin bounce-back; and
determining an edema result based on the characteristics of the skin bounce-back.

54. The method of claim 53, further comprising instructing the user to depress the skin area with a finger or a probe.

55. The method of claim 53, further comprising calculating an edema score.

56-57. (canceled)

58. The method of claim 53, further comprising compensating for an elapsed time period during which the skin area is obstructed, wherein the skin area is obstructed by (i) a finger of the user, (ii) a probe, or (iii) both (i) and (ii).

59-62. (canceled)

63. The method of claim 53, further comprising measuring a perfusion index and correlating the perfusion index with the analyzing of the one or more images to confirm the edema result.

64-77. (canceled)

78. A system for determining an edema result, the system comprising:
a probe having a proximal end and a distal end, the distal end having a distal surface for causing a temporary indentation in a skin surface of a subject; and
an electronic device including
a housing for enclosing internal components, the housing having an external surface on which the proximal end of the probe is removably attached,
an electronic interface configured to receive user input from a user and to provide user instructions, the user input including a user request for an edema test, the user instructions including procedural steps for the edema test;
a camera for detecting a change in skin characteristics of the subject;
a memory storing machine-readable instructions; and
a control system including one or more processors configured to execute the machine-readable instructions to activate the edema test,
upon activation of the edema test, provide the user instructions including locating via the camera the skin area in the subject for testing,
instruct the user to depress the skin area for causing the temporary indentation,
capture, via the camera, one or more images of the temporary indentation as the skin area bounces back to a full or partial undepressed state,
analyze the one or more images for characteristics of skin bounce-back, and
determine an edema result based on the characteristics of the skin bounce-back.

79. The system of claim 78, wherein the characteristics of the skin bounce-back include a length of time elapsing between causing the temporary indentation and achieving the full or partial undepressed state, (ii) a skin color change during the skin bounce-back, or (iii) both (i) and (ii).

80. (canceled)

81. The system of claim 78, wherein the probe has an imaging conduit that is at least in part transparent or translucent, the imaging conduit being in visual communication with the camera.

82. The system of claim 78, wherein the distal surface includes one or more protrusions for causing the temporary indentation.

83. The system of claim 78, wherein the probe includes a lens at the distal end, the lens being in visual communication with the camera.

84. The system of claim 78, wherein the one or more processors are further configured to execute the machine-readable instructions to overlay an augmented reality (AR) indicia over a body part of the subject, the augmented reality (AR) indicia locating the skin area for testing.

85. The system of claim 78, wherein the distal end is movable relative to the proximal end, the distal end being supported by a biasing element to exert a test pressure on the skin area.

86-87. (canceled)
